(19) United States
(12) Patent Application Publication
(10) Pub. No.: US 2004/0030391 A1
(43) Pub. Date: Feb. 12, 2004

(54) Artificial intervertebral disc spacers

(76) Inventor: Bret Ferree, Cincinnati, OH (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/422,282

(22) Filed: Apr. 24, 2003

Related U.S. Application Data
(60) Provisional application No. 60/375,212, filed on Apr. 24, 2002.

(51) Int. Cl. A61F 2/44

(52) U.S. Cl. 623/17.16; 623/17.15

(57) ABSTRACT

Intervertebral disc spacer components include spherical articulating surfaces to promote more natural spinal movement between one or both vertebrae. The invention broadly encompasses the provision of a single artificial disc replacement end plate (ADR EP) with a bi-convex disc spacer where the maximum vertical distance of the spacer is centrally located or a disc spacer with two or more components, wherein where at least one of the articulations between spacer components is spherical (a generally convex articulating surface mated with a generally concave articulating surface). The invention may further include the step of shaping the vertebral endplates to improve the articulation between the disc spacer and the vertebra by increasing the surface contact between the disc spacer and the vertebra. Although described in terms of artificial disc replacements, the apparatus and methods are applicable to other areas of the body, including knee replacements.

### REFERENCE TO RELATED APPLICATION

This application claims priority from U.S. Provisional Patent Application Serial No. 60/375,212, filed Apr. 24, 2002, the entire content of which is incorporated herein by reference.

### FIELD OF THE INVENTION

This invention relates generally spine surgery and, in particular, to artificial intervertebral disc spacers.

### BACKGROUND OF THE INVENTION

Spacers, usually constructed of metal, have been used to treat unicompartmental arthritis of the knee. In such cases, a knee prosthesis is placed in the arthritic joint spacer. The surfaces of the spacer are shaped to articulate with the femoral condyle and the tibia plateau.

Intervertebral prosthetic discs are also known. One example is presented in U.S. Pat. No. 5,888,226, which teaches the use of disc spacers with convex vertebral surfaces, where the maximum vertical dimension is non-central. This reference also teaches the use of flat articulating surfaces between disc spacer components. Another example is given in U.S. Pat. No. 4,348,921, which discloses the use of a one- or two-piece disc spacer. The two-component disc spacer may articulate through a spherical joint.

The device described in the '921 patent is designed to avoid movement between the disc spacer and the vertebrae. The disclosure describes corrugations or projections to improve the “friction fit”. A flange is includes that would further inhibit movement between the disc spacer and the vertebrae, and the specification mentions that it is important that the prosthesis does not allow lateral movement.

FIG. 1 is a lateral view of the spine and a prior-art artificial disc replacement (ADR) spacer. FIG. 2 is a view of the lateral aspect of the spine and another prior-art disc spacer which is textured to inhibit motion between the spacer and the vertebrae. Based upon such approaches, the need remains for disc spacer component device that permits more natural spinal movement.

### SUMMARY OF THE INVENTION

This invention improves upon the prior art by providing disc spacer components including spherical articulating surfaces to promote more natural spinal movement. Depending upon the embodiment, spherical articulations may be permitted between one or both vertebrae. Such articulations may occur between the vertebrae and two pairs of spherical articulating surfaces between the disc spacer components, or between the vertebrae and one pair of spherical articulating surfaces between the disc spacer components.

The invention broadly encompasses the provision of a single artificial disc replacement end plate (ADR EP) with a bi-convex disc spacer where the maximum vertical distance of the spacer is centrally located or a disc spacer with two or more components, wherein where at least one of the articulations between spacer components is spherical (a generally convex articulating surface mated with a generally concave articulating surface).

Components according to the invention may be composed of any biologically acceptable material including chrome cobalt, titanium, polyethylene, Nitinol, ceramic, stainless steel, and polymers, including elastomers. At least one component may have elastic or spring-like properties.

The invention may further include the step of shaping the vertebral endplates to improve the articulation between the disc spacer and the vertebra by increasing the surface contact between the disc spacer and the vertebra. Although described in terms of artificial disc replacements, the apparatus and methods are applicable to other areas of the body, including knee replacements.

### BRIEF DESCRIPTION OF THE DRAWINGS

FIG. 1 is a lateral view of the spine and a prior art disc spacer ADR;

FIG. 2 is a view of the lateral aspect of the spine and another prior art disc spacer;

FIG. 3A is a lateral view of the disc spacer of the present invention;

FIG. 3B is a lateral view of the embodiment of the device shown in FIG. 3A;

FIG. 4A is a view of the top of the disc spacer shown in FIG. 3A;

FIG. 4B is a view of the top of an alternative shape of the disc spacer shown in FIG. 4A;

FIG. 5A is a sagittal cross section of the disc spacer shown in FIG. 3A;

FIG. 5B is a view of the top of the central component in the embodiment of the device shown in FIG. 5A;

FIG. 5C is a view of the top of an alternative central component to that shown in FIG. 5B;

FIG. 5D is a view of the top of an alternative central component to that shown in FIG. 5C;

FIG. 5E is a view of the lateral aspect of the spine and the embodiment of the device shown in FIG. 5;

FIG. 5F illustrates how depressions may be used in lieu of a through-hole;

FIG. 6A is a lateral view of an alternative embodiment of the disc spacer;

FIG. 6B is a lateral view of the embodiment of the spacer shown in FIG. 6A;

FIG. 7 is a sagittal cross section of the embodiment of the device shown in FIG. 6A;

FIG. 8A is a view of the lateral aspect of an alternative embodiment of the disc spacer;

FIG. 8B is a sagittal cross section of the embodiment of the disc spacer shown in FIG. 8A;

FIG. 9A is a sagittal cross section of an alternative embodiment of the device;

FIG. 9B is a lateral view of the embodiment of the disc spacer shown in FIG. 9A;

FIG. 9C is a lateral view of an alternative embodiment of the device shown in FIG. 9B;

FIG. 9D is a lateral view of a flexed spine and the embodiment of the device shown in FIG. 9C;

FIG. 10A is sagittal cross section of the spine and an alternative embodiment of the device;

FIG. 10B is a sagittal cross section of the spine and an alternative embodiment of the device shown in FIG. 10A;

FIG. 11 is a sagittal cross section of the spine and an alternative embodiment of the disc spacer;

FIG. 12 is a sagittal cross section of an alternative embodiment of the device drawn in FIG. 9A;

FIG. 13 is a sagittal cross section of an alternative embodiment of the device shown in FIG. 5A;

FIG. 14 is a sagittal cross section of an alternative embodiment of the device shown in FIG. 13; and

FIG. 15 is sagittal cross section of the knee and an example of an embodiment of the device designed for use in the knee.

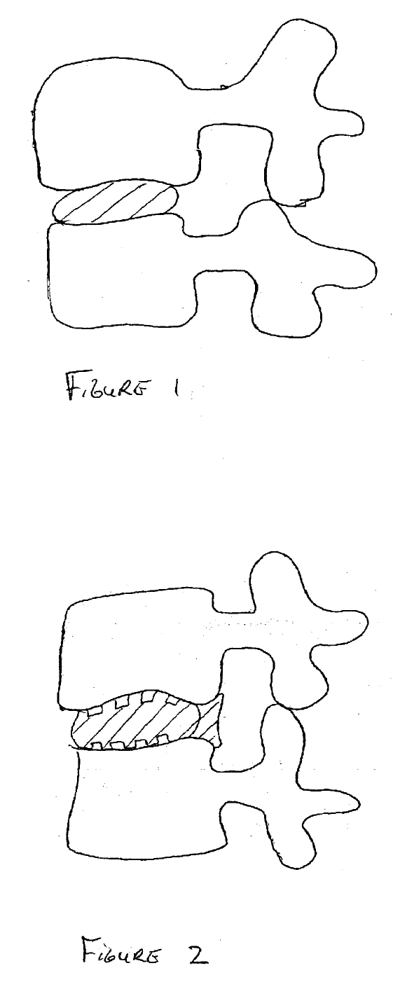

### DETAILED DESCRIPTION OF THE INVENTION

The present invention is a spacer that can be placed into the disc space. The spacer is not fixed to either vertebra. Rather, the prosthesis is free to self-center with spinal movement. The spacer also provides at least a certain degree of distraction. The prosthesis can be made of metal, a polymer such as polyethylene, or ceramic, or any combination thereof, such as metal/polyethylene, metal/ceramic, ceramic/polyethylene, and so forth. The articulated spacers disclosed herein may also be used in the knee.

Shape memory properties of the material may be helpful. The spacer is generally bioconvex in shape to closely match the concavities of the vertebrae.

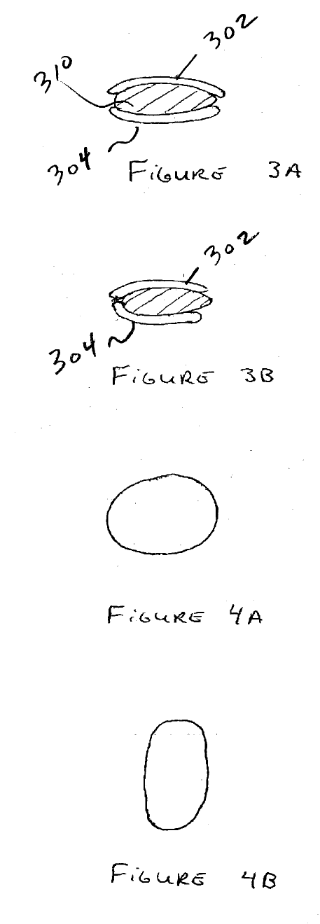

Reference is again made to the drawings, wherein FIG. 3A provides a lateral view of a disc spacer according to the invention including three components, namely two outer convex components 302, 304, and a center component 310. The surfaces of the outer, convex, components 302, 304 are polished to facilitate articulation with the vertebrae and the center component 310. FIG. 3B is a lateral view of the embodiment of the device drawn in FIG. 3A, with the outer components 302, 304 seen in a forward-most position. Movement between these disc spacer components facilitates more natural spinal motion.

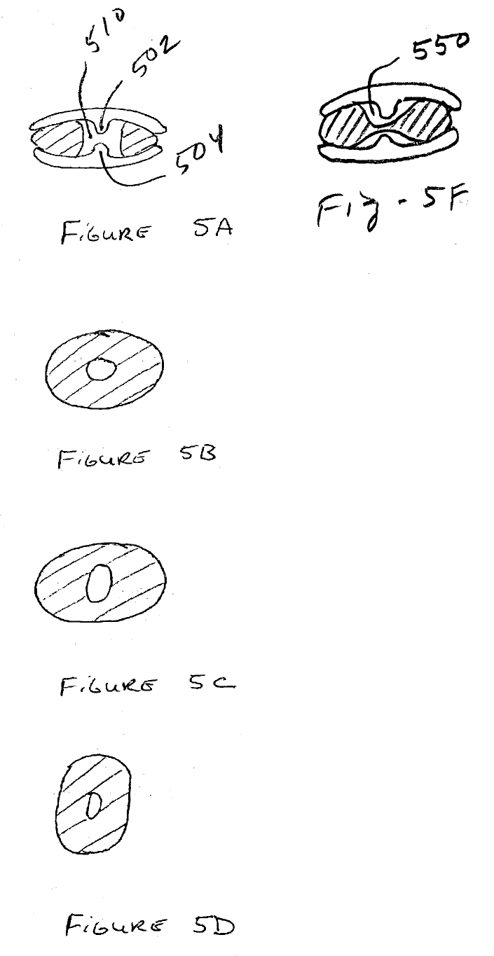

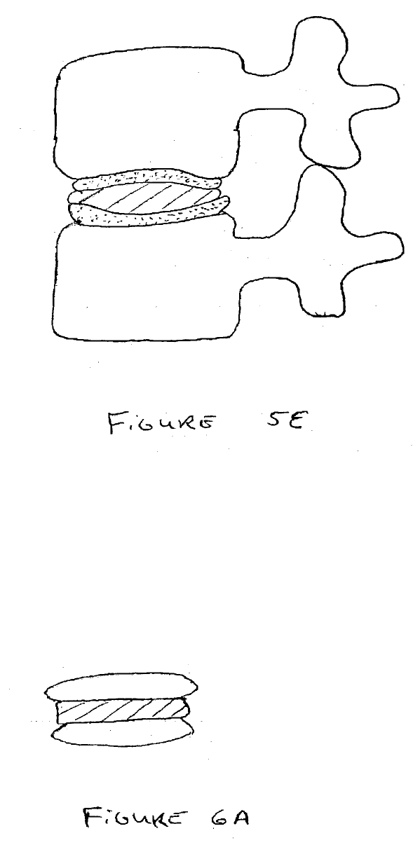

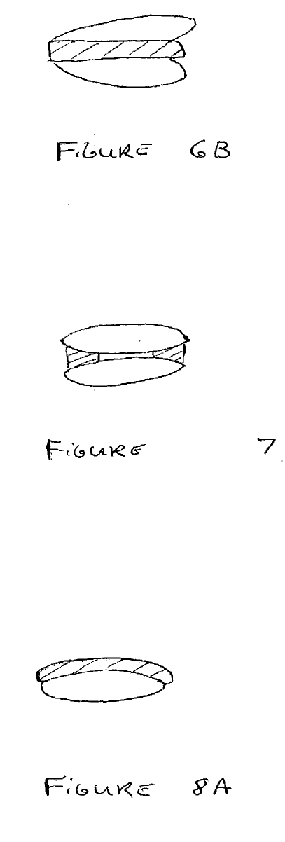

FIG. 4A is a view of the top of the disc spacer drawn in FIG. 3A. FIG. 4B is a view of the top of an alternative shape of the disc spacer drawn in FIG. 4A. FIG. 5A is a sagittal cross section of the disc spacer drawn in FIG. 3A, perhaps better illustrating how the disc spacer articulates with the vertebrae above and below the disc spacer. The disc spacer components also articulate through generally convex and concave articulating surfaces above and below the central disc spacer component. Thus, the disc spacer articulates through four different locations. Projections 502, 504 from the outer components fit within a hole 504 in the inner, central, component. As shown in FIG. 5E, depressions 550 may be used in lieu of a through-hole.

In all cases cooperation between the components prevents dissociation of the spacer. The outer components could reversibly flatten in response to axial loads. The outer components could have elastic properties much like the components described in my co-pending U.S. patent application Ser. No. 60/399,876, incorporated herein by reference.

FIG. 5B is a view of the top of the central component in the embodiment of the device drawn in FIG. 5A. FIG. 5C is a view of the top of an alternative central component to that drawn in FIG. 5B, wherein the central opening is oval. FIG. 5D is a view of the top of an alternative central component to that drawn in FIG. 5C, wherein the component is oval.

FIG. 5E is a view of the lateral aspect of the spine and the embodiment of the device drawn in FIG. 5A. Note that movement between the disc spacer and the vertebrae allows the disc spacer to “self-center.” For example, the disc spacer may move forward during spinal extension. Movement also occurs through the two articulations between the three disc spacer components.

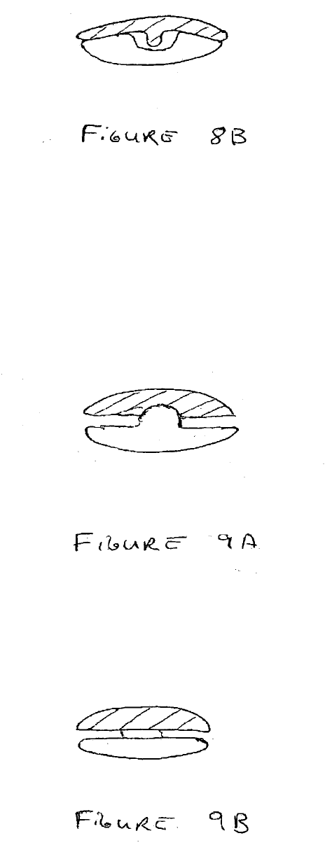

FIG. 6A is a lateral view of an alternative embodiment of the disc spacer. The disc spacer has three components. FIG. 6B is a lateral view of the embodiment of the spacer drawn in FIG. 6A. The outer components are drawn in a tilted position. Tilting of the outer components facilitates spinal motion.

FIG. 7 is a sagittal cross section of the embodiment of the device drawn in FIG. 6A. The disc spacer articulates with the vertebra above and below the spacer. The outer disc spacer components also articulate with the central, bi-concave, disc spacer component. As described in conjunction with FIG. 4, the disc spacer may be circular or oval.

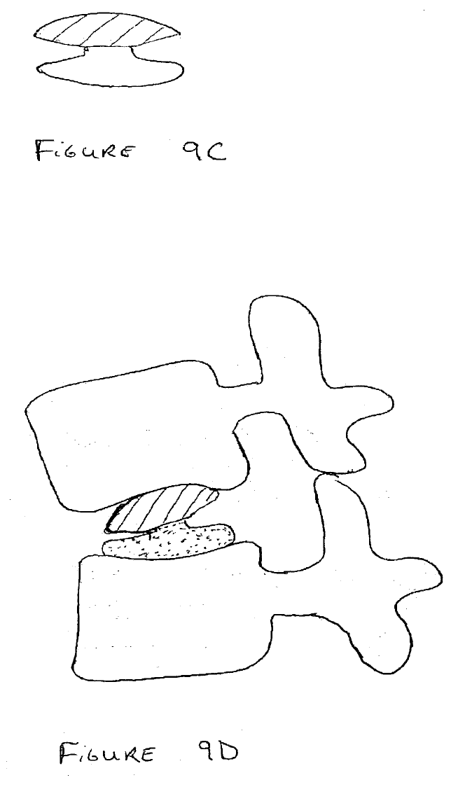

FIG. 8A is a view of the lateral aspect of an alternative embodiment of a disc spacer incorporating two components. FIG. 8B is a sagittal cross section of the embodiment of the disc spacer drawn in FIG. 8A. Both components articulate with the vertebra above and below the disc spacer. The spacer components also articulate with each other through generally convex and generally concave articulating surfaces. A locking projection similar to that shown in FIG. 5A, is used to prevent the components from dissociating.

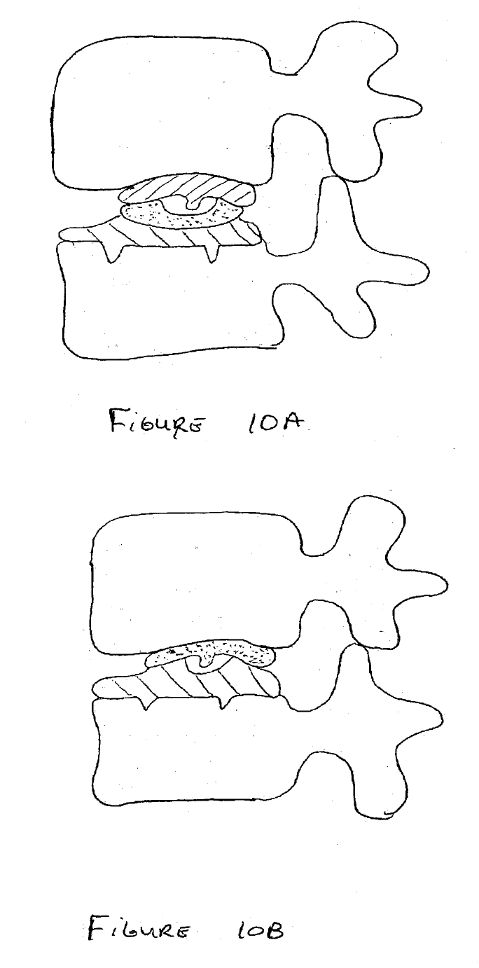

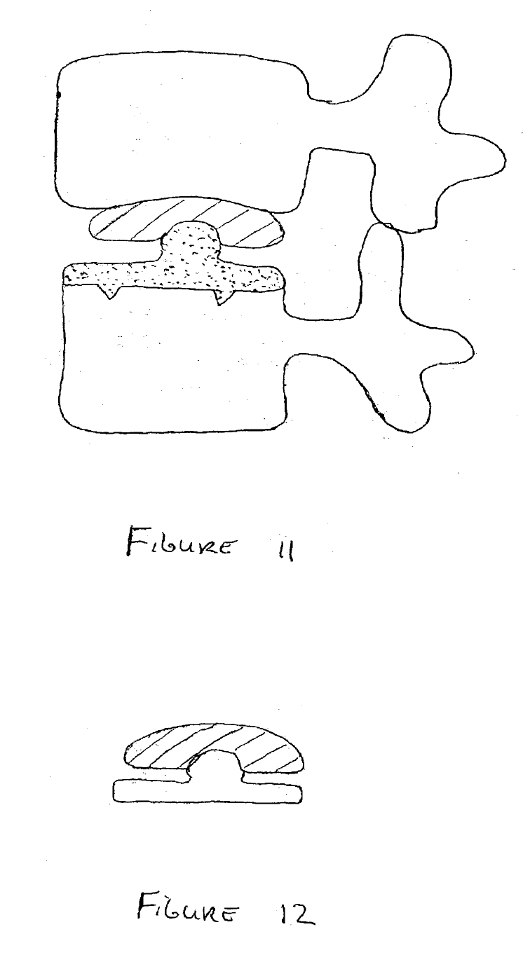

FIG. 9A is a sagittal cross section of an alternative embodiment of the device. The spacer has two components. Both components articulate with each other and the vertebrae. FIG. 9B is a lateral view of the embodiment of the disc spacer drawn in FIG. 9A. FIG. 9C is a lateral view of an alternative embodiment of the device drawn in FIG. 9B. The components are shaped to increase the motion across the articulation between the disc spacer components. FIG. 9D is a lateral view of a flexed spine and the embodiment of the device drawn in FIG. 9C.

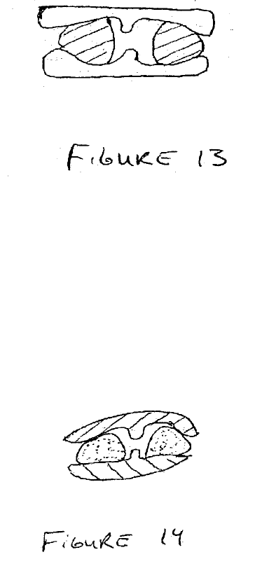

FIG. 10A is sagittal cross section of the spine and an alternative embodiment of the device. Articulating components similar to those drawn in FIG. 8B articulate with a single ADR endplate. The ADR endplate (EP) is press fit or otherwise connected to the vertebra. Preferably, the ADR EP does not move relative to the vertebra (EP). FIG. 10B is a sagittal cross section of the spine and an alternative embodiment of the device drawn in FIG. 10A. The inferior articulating component is connected to the vertebra much like an ADR EP is connected to the spine. Articulation is limited to the two disc spacer components and the superior component and the vertebra superior to the disc spacer. As illustrated in the figure, the disc spacer may articulate with only a portion of the vertebral EP.

FIG. 11 is a sagittal cross section of the spine and an alternative embodiment of the disc spacer. The inferior component of the disc spacer drawn in FIG. 9A is attached to the vertebra below the disc spacer. FIG. 12 is a sagittal cross section of an alternative embodiment of the device drawn in FIG. 9A. The inferior surface of the inferior disc spacer component is flat. The flat surface of the disc spacer component facilitates translation of the vertebrae.

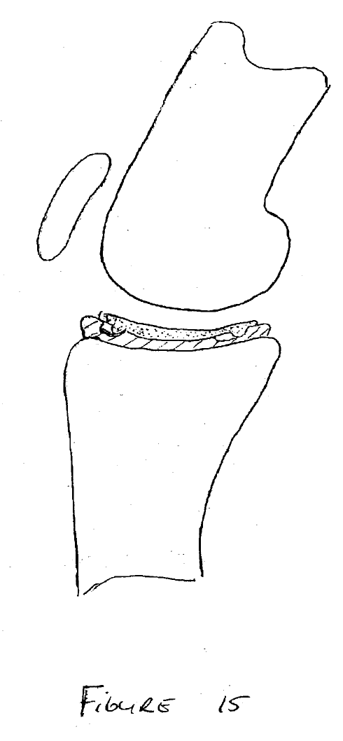

FIG. 13 is a sagittal cross section of an alternative embodiment of the device drawn in FIG. 5A. The vertebral surfaces of both disc spacer components are flat. The vertebral EPs could be shaped to improve the surface contact between the disc spacer and the vertebrae. FIG. 14 is a sagittal cross section of an alternative embodiment of the device drawn in FIG. 13. The articulating surfaces on one side of the central component are flat. The flat articulating surfaces facilitate translation.

FIG. 15 is sagittal cross section of the knee and an example of an embodiment of the device designed for use in the knee. The two component device articulates with the tibia and the femur. The two components also articulate with each other. All of the articulating surfaces are highly polished. Alternatively, the surfaces of the spacer components that articulate with the bones of the knee could be less polished to encourage movement between the spacer components rather than movement between the spacer and the bones. In particular, it may be beneficial to discourage movement between the spacer and the tibia. The spacer may be coupled with a device described in my co-pending U.S. patent application Ser. No. 60/376,505, incorporated herein by reference. The devices taught in this other application also prevent extrusion of articulating devices from the joints of the body.

I claim:

1. A disc spacer, comprising:
a device that articulates with respect to:
a) at least one spherical interface involving convex and concave surfaces, and
b) at least one vertebral endplate.
